(19) United States
(12) Patent Application Publication
(10) Pub. No.: US 2023/0207048 A1
(43) Pub. Date: Jun. 29, 2023

(54) SOMATIC COPY NUMBER VARIATION DETECTION

(71) Applicant: ILLUMINA, INC., lllumina way, CA (US)

(72) Inventors: Han-Yu Chuang, San Diego, CA (US); Chen Zhao, San Diego, CA (US)

(21) Appl. No.: 16/333,933

(22) PCT Filed: Sep. 21, 2017
(86) PCT No.: PCT/US2017/052766

§ 371 Date: Mar. 15, 2019

Related U.S. Application Data
(60) Provisional application No. 62/447,065, filed on Jan. 17, 2017.
(60) Provisional application No. 62/398,354, filed on Sep. 22, 2016.

(51) Int. Cl. G16B 20/10; G16B 20/20; G16B 30/10

(52) U.S. Cl. CPC G16B 20/10; G16B 20/20; G16B 30/10

(57) ABSTRACT

Presented herein are techniques for assessing copy number variation. The techniques include generating a baseline representative of or mimicing a hypothetical matched sample for an individual biological sample from a set of baseline samples that are not matched to the biological sample. Normalized sequencing data from the set of baseline samples that includes at least one copy number baseline for a region of interest is provided to a user.

### CROSS REFERENCE TO RELATED APPLICATIONS

This application claims priority to and the benefit of International Application No. PCT/US2017/052766, filed on Sep. 21, 2017, which claims priority to and the benefit of U.S. Provisional Application No. 62/398,354, entitled “SOMATIC COPY NUMBER VARIATION DETECTION” and filed Sep. 22, 2016, and to U.S. Provisional Application No. 62/447,065, entitled “SOMATIC COPY NUMBER VARIATION DETECTION” and filed Jan. 17, 2017, the disclosures of which are incorporated herein by reference for all purposes.

### BACKGROUND

The present disclosure relates generally to the field of data related to biological samples, such as sequence data. More particularly, the disclosure relates to techniques for determining copy number variation based on sequencing data.

Genetic sequencing has become an increasingly important area of genetic research, promising future uses in diagnostic and other applications. In general, genetic sequencing involves determining the order of nucleotides for a nucleic acid such as a fragment of RNA or DNA. Some techniques involve whole genome sequencing, which involves a comprehensive method of analyzing a genome. Other techniques involve targeted sequencing of a subset of genes or regions of the genome. Targeted sequencing focuses on regions of interest, generating a smaller and more compact data set. Further, targeted sequencing reduces sequencing costs and data analysis burdens while also allowing deep sequencing at high coverage levels for detection of variants in the regions of interest. Examples of such variants may include somatic mutations, single nucleotide polymorphisms, and copy number variations. Detection of variants may provide clinicians with information about disease likelihood or susceptibility. Accordingly, there is a need for improved detection of variants in sequencing data.

### BRIEF DESCRIPTION

The present disclosure provides a novel approach for detection of copy number variations in a biological sample. As provided herein, copy number variations (CNVs) are genomic alterations that result in an abnormal number of copies of one or more genomic regions. Structural genomic rearrangements such as duplications, multiplications, deletions, translocations, and inversions can cause CNVs. Like single-nucleotide polymorphisms (SNPs), certain CNVs have been associated with disease susceptibility. The term “copy number variation” herein may refer to variation in the number of copies of a nucleic acid sequence present in a test sample of interest in comparison with an expected copy number. For example, for humans, the expected copy number of autosome sequences (and X chromosome sequences in females) is two. Other organisms may have different expected copy numbers according to their genomic structure. Copy number variation may be the result of duplication or deletion. In certain embodiments, copy number variants refer to sequences of at least 1 kb that are duplicated or deleted. In one embodiment, copy number variants may be at least a single gene in size. In another embodiment, copy number variants may be at least 140bp, 140-280bp, or at least 500bp.

In one embodiment, a “copy number variant” refers to the sequence of nucleic acid in which copy-number differences are found by comparison of a sequence of interest in test sample with an expected level of the sequence of interest. As provided herein, a reference sample is derived from a set of sequencing data of unmatched samples to generate normalization information that permits an individual test sample to be normalized such that deviations from expected copy numbers may be determined on normalized sequencing data. The normalization data is generated using the techniques provided herein and permits normalization to a hypothetical most representative sample matched to the test sample. By normalizing the test sample, noise introduced by sequencing or other bias is removed.

In certain embodiments, the raw sequencing data coverage from a targeted sequencing run is normalized to reduce technical and biological noise to improve CNV detection. In one embodiment, samples of interest (e.g., fixed formalin paraffin embedded samples) are sequenced according to a desired sequencing technique, such as a targeted sequencing technique that uses a sequencing panel of probes to target regions of interest. Once the sequencing data is collected, the sequencing data is normalized to remove noise, and the normalized data is subsequently analyzed to detect CNVs.

In one embodiment, a method of normalizing copy number is provided that includes the steps of receiving a sequencing request from a user to sequence one or more regions of interest in a biological sample; acquiring baseline sequencing data from the one or more regions of interest from a plurality of baseline biological samples that are not matched to the biological sample; determining copy number normalization information using the baseline sequencing data, wherein the copy number normalization information comprises at least one copy number baseline for a region of interest of the one or more regions of interest; and providing the copy number normalization information to the user.

In another embodiment, a method of detecting copy number variation is provided that includes the steps of acquiring sequencing data from a biological sample, wherein the sequencing data comprises a plurality of raw sequencing read counts for a respective plurality of regions of interest; and normalizing the sequencing data to remove region-dependent coverage. The normalizing comprises: for each region of interest, comparing a raw sequencing read count of one or bins in a region of interest of the biological sample to a baseline median sequencing read count to generate a baseline-corrected sequencing read count for the one or more bins in the region of interest, wherein the baseline median sequencing read count for one or more bins in the region of interest is derived from a plurality of baseline samples that are not matched to the biological sample and is determined from only the most representative portions of the baseline sequencing data for each region of interest; and removing GC bias from the baseline-corrected sequencing read count to generate a normalized sequencing read count for each region of interest. The method also includes determining copy number variation in each region of interest based on the normalized sequencing read count of the one or more bins in each region of interest.

In another embodiment, a method of assessing a targeted sequencing panel is provided that includes the steps of identifying a first plurality of targets in a genome for a targeted sequencing panel, wherein the first plurality of targets corresponds to portions of a respective plurality of genes; determining a GC content of each of the first plurality of targets; eliminating targets of the first plurality of targets with GC content outside of a predetermined range to yield a second plurality of targets smaller than the first plurality of targets; when, after the eliminating, the an individual gene has fewer than a predetermined number of targets corresponding portions to the individual gene, identifying additional targets in the individual gene; adding the additional targets to the second plurality to yield a third plurality of targets; and providing a sequencing panel comprising probes specific for the third plurality of targets.

### DRAWINGS

FIG. 1 is a diagrammatical overview of methods for detecting copy number variants in accordance with the present techniques;

FIG. 2 is a block diagram of a sequencing device that may be used in conjunction with the methods of FIG. 1;

FIG. 3 is a schematic overview of an example of the normalization technique in accordance with embodiments of the disclosure;

FIG. 4 shows bin profile data for sequencing results before and after normalization, as provided herein;

FIG. 5 shows noise present in normal FFPE samples relative to a highly degraded cell line and a normal cell line mixture;

FIG. 6 is a panel of plots showing that baseline correlation is poor among different sample types;

FIG. 7 shows examples of one or more types of bin filtering that may be applied to baseline reference sequencing data from non-matched samples to remove bad bins to generate baselines for normalization;

FIG. 8 shows hierarchical clustering to identify representative baselines using baseline reference sequencing data from non-matched normal samples;

FIG. 9 shows the results of baseline correction with linear regression to remove noise, whereby c1 and c2 are two representative baselines learned from hierarchical clustering

FIG. 10 shows variable and sample-dependent GC bias among samples S1, S2, S3, and S4;

FIG. 11 shows normalization that includes baseline and GC bias correction using input data A and yielding corrected data in plot D, whereby A to B represents linear regression using baselines of the trained algorithm and B to C represents generating a fitted curve representative of GC bias for the sample, and C to D represents flattening the fitted curve to remove the GC bias from the sample;

FIG. 12 shows before and after normalization results, including sequence bins for ERBB2;

FIG. 13 shows that fold change detection is stable independent of baseline used, with R²=0.99 across 340 FFPE samples;

FIG. 14 shows high concordance between the normalization techniques as provided herein and ddPCR across 22 FFPE samples tested using a panel for a number of regions of interest, including EGFR, ERBB2, FGFR1, MDM2, MET, and MYC;

FIG. 15 shows a comparison of results using the normalization techniques as provided herein and a control free sample for EGFR;

FIG. 16 shows a median absolute deviation comparison of results using the normalization techniques as provided herein and matched normal samples with a paired t test p-value of 0.0202,

FIG. 17 shows fold change comparison, with detected fold change (FC) comparison between the normalization techniques as provided herein (y-axis) and matched normal (x-axis);

FIG. 18 shows KIT variants detected using normalization techniques as provided herein;

FIG. 19 shows KIT variants detected using an alternate principal components analysis technique;

FIG. 20 shows BRCA2 variants detected using normalization techniques as provided herein;

FIG. 21 shows BRCA2 variants failed to be detected using an alternate principal components analysis technique;

FIG. 22 is a schematic representation of probe design for example genes showing bin regions;

FIG. 23 is a schematic representation of bin counts based on fragments, not reads;

FIG. 24 is table of bin designations and characteristics;

FIG. 25 is a plot of target size distribution for a probe;

FIG. 26 shows gene median absolute distribution and comparison to number of targets and GC content of targets;

FIG. 27 shows gender classification of FFPE samples and presence of chromosome Y coverage;

FIG. 28 shows a comparison of probe coverage with and without coverage enhancers;

FIG. 29 shows a summary of probe coverage for a variety of genes; and

FIG. 30 shows an example of a graphical user interface of detected copy number variation.

### DETAILED DESCRIPTION

The present techniques are directed to analysis and processing of sequencing data for improved somatic copy number variation (CNV) detection. CNV detection is often confounded by various types of bias introduced during sample preservation, library preparation, or sequencing. Without bias, read depth/coverage should be uniform across the genome for diploid regions, and proportionally higher (lower) for copy number gain (loss) regions. With bias, this assumption is no longer valid, at least for regions of the genome that are subject to bias. Removal of bias or normalizing the data first, e.g., prior to CNV detection, achieves more accurate CNV calling as provided herein.

Provided herein are techniques that generate a reference baseline for an individual biological sample that is useful for normalizing the sequencing date before assessing variations that are representative of copy number changes for one or more regions of interest in a genome. The disclosed techniques provide reference or normalization information without relying on a matched sample from the individual from whom the test sample is obtained to normalize a test sample. While other techniques may use the patient’s own tissue to generate the reference, using a matched sample taken from the same individual as the biological sample presents certain challenges. For example, variation in sample collection (sample quality, selected tissue sites) may mean that reference sample is not truly representative of normal tissue. Further, insofar as the introduction of bias that influences sequencing data may vary between samples, the matched reference sample may have a different level of introduced bias relative to the test sample, which in turn may lead to inaccuracies and inadequately normalized data. In addition, not all test samples have available matched tissue or matched tissue of sufficiently high quality for sequencing.

Accordingly, the disclosed techniques facilitate more accurate copy number variation assessment by generating normalization information with reduced bias and without using a matched sample. The normalization information may be used to normalize a set of sequencing data prior to CNV detection in the individual sample. The normalization information is generated using a set or pool of unmatched reference baseline biological samples. Sequencing data generated from this set is then used to generate normalization information that is representative of a most typical hypothetical matched reference sample. That is, the normalization information represents a virtual calibrated gold standard reference against which any individual test sample may be normalized against.

In certain embodiments, CNVs may be detected using whole genome sequencing techniques. However, such techniques are expensive and involve generating data that may be outside the regions of interest. In other embodiments, using targeted sequencing techniques to detect CNVs is less expensive and is associated with a faster turnaround time. In targeted sequencing, the targeted probes are used to pull down regions of interest from the sample DNA for sequencing; the probes used may vary depending on the regions of interest and the desired detection outcome. However, the coverage of sequencing data from a targeted sequencing run may be variable due to varying characteristics of the regions of interest (e.g., the target sequences) in the genome, the probes, and the quality of the sample itself. For example, probes specific for larger targets (e.g., longer exons) will typically have more reads or coverage than probes for smaller targets. In another example, degraded areas of the DNA in a biological sample will have fewer reads. In yet another example, GC-rich or GC-poor regions of interest will have variations in coverage that may be nonlinear. Accordingly, variability in coverage for sequencing data from targeted sequencing runs may introduce noise that interferes with the accuracy of CNV detection based on coverage/read depth.

Table 1 illustrates the common types of sequencing bias/noise present in enrichment data. For example, different probes may have different pull-down efficiency, thereby creating uneven coverage across different regions (baseline effect). Coverage might also be GC dependent - regions with low or high GC content have lower coverage in general. Additionally, coverage might be affected by formalin-fixed paraffin-embedded (FFPE) sample quality or sample type. All of the aforementioned artifacts present challenge for amplification detection. CNV Robust Analysis aims to remove these biases (i.e., using data normalization) before CNV calling.

TABLE 1

Sources of bias in biological samples

| | |
|---|---|
| SOURCE OF BIAS | EXPLANATION |
| Sequencing depth | Sample to sample variation |
| Target size | Larger targets attract more reads |
| PCR duplicates | Read level |
| Probe pull down efficiency | Sequence content specific |
| GC bias | Target specific, non-linear effect |
| DNA quality | Degradation |

The disclosed techniques leverage a panel of reference normal samples to remove the need to use a matched normal sample in read count normalization of a tumor sample. Specifically, sequence read count bias is strongly correlated to tissue type and DNA quality of a test sample, with the equivalent impact as the germline genetics of the sample if not even stronger. Therefore, with a good variety of reference normal samples representing different tissue types and different DNA quality, CRAFT in silicon assembles a “virtual” matched normal sample to a test tumor sample through a linear combination of all the reference normal samples.

The panel of reference normal samples goes through a data-driven clustering process to form read count baselines. Each reference baseline is a representative of certain tissue type, DNA quality, and other systematic background on read count bias, rather than the true copy number changes in a genome. For a test sample, a linear regression of the reference baselines is performed against the sample read count data to determine the coefficient of each baseline. Each test sample results in a unique set of coefficients, mimicking a virtual matched normal sample. When a user acquires sequencing data with the particular sequencing panel, the user can normalize the acquired sequencing data using the coefficients. In one embodiment, coefficients may be applied via a linear combination to yield a weighted copy number value for a particular region of interest (e.g., a gene).

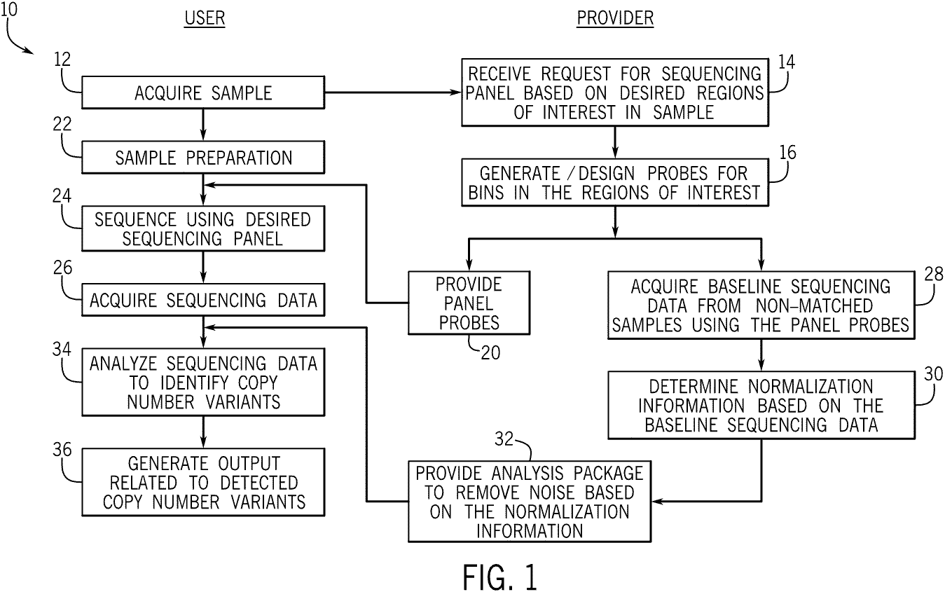

To that end, the disclosed techniques eliminate or reduce copy number variation assessment errors that result from sequencing bias. FIG. 1 is a flow diagram 10 showing interactions between end user and providers using the normalization techniques as provided herein. The depicted flow diagram 10 is presented in the context of a targeted sequencing panel. However, it should be understood that similar interactions may also occur in the context of a whole genome sequencing reaction.

At step 12, a user acquires a biological sample of interest for assessment. The biological sample may be a tissue sample, fluid sample, or other sample containing at least a portion of a genome or genomic DNA. In certain embodiments, the biological sample is fresh, frozen, or preserved using standard histopathological preservatives such as FFPE. The biological sample may be a test sample or may be an internal sample used to generate the normalization information. In embodiments in which the biological sample is assessed using a targeted sequencing panel, the user transmits a targeting sequencing request to a provider, whereby the request includes a selected pre-existing sequencing panel and/or a customized sequencing panel based on desired regions of interest in the genomic DNA of the sample. The request may include customer information, biological sample organism information, biological sample type information (e.g. information identifying whether the sample is fresh, frozen, or preserved), tissue type, and desired sequencing assay type. The request may also include nucleic acids sequences for desired probes of a sequencing panel and/or nucleic acid sequences of regions of interest in a genome that may be used by the provider to design and/or generate probes for a targeted sequencing panel.

The provider receives the request at step 14 and designs and/or generates probes to be used in the sequencing based on the designated probe set and/or the designated regions of interest (e.g., bins) at step 16. In certain embodiments, for pre-existing sequencing panels, the probes may be generated and kept in inventory before the request is received at step 14. The probes are provided to the user at step 20 and, subsequent to any relevant sample preparation at step 22, used to sequence the biological sample at step 24. The user acquires sequencing data from the sequencing at step 26.

When the user selects probes for a targeted sequencing panel, the probes are also used in a baseline sequencing reaction on a set of non-matched samples (e.g., other biological samples that are not matched to or from the same individual as the biological sample) to acquire baseline sequencing data at step 28. The baseline sequencing data is used to generate normalization information at step 30, which is provided to the user at step 32. Using the normalization information, the user normalizes the sequencing data of the test sample and subsequently analyzes the acquired sequencing data of the biological sample at step 34 to identify copy number variants for locations that are included in the targeted sequencing panel. That is, in the context of a targeted sequencing panel, which facilitates sequencing of only a portion of the genome, only copy number variants present in the sequenced portion can be identified. This is in contrast to whole genome applications is which copy number variants throughout the entire genome may be identified according to the present techniques.

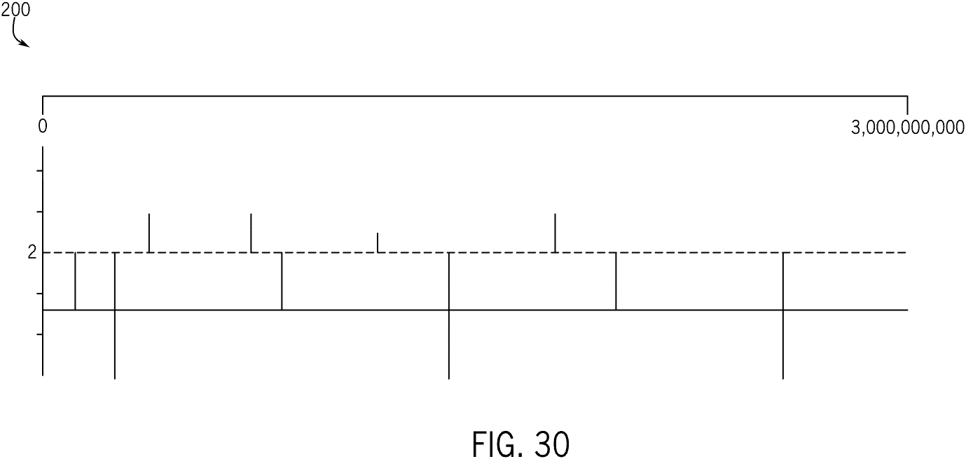

In response to identifying the copy number variants, an output may be provided to the user at step 36. The output may include a displayed graphical user interface (see FIG. 30) that includes graphical icons of copy number at particular locations in the genome.

The user may be an external or internal user of sequencing services of the provider. For example, the steps of the flow diagram 10 may be performed as a part of calibrating or generating any new targeted sequencing panel product, which may also include an external request for a customized sequencing panel. A given targeted sequencing panel will be associated with particular bias tendencies based on the regions of interest targeted by the panel probes. This bias may interfere with accurate assessment of copy number variation. Accordingly, the steps of the flow diagram 10 may be performed when any targeted sequencing panel that includes a set of probes is designed, modified, or updated. In another embodiment, if a user request includes regions of interest in a genome, a panel including a set of probes may be generated and evaluated using the disclose techniques to yield normalization information. The normalization information may be evaluated using a set of metrics. If the metrics indicate that the panel yields poor normalization information, the panel may be discarded and the probes redesigned (e.g., shifted 50 bp in either direction). The new probes may be tested using the steps of the flow diagram 50 until high quality normalization information is obtained. In one embodiment, the metrics are obtained by applying the normalization information before identifying copy number variants in an internal sample. If the identified copy number variants across the sequenced regions deviate from an expected distribution, an output may be provided indicating that a new sequencing panel (e.g., a probe redesign) should be triggered. The expected distribution may be associated with a likely distribution of copy number variants. For example, most variants are within a two or three-fold change in either direction. If the internal sample is shown to have a larger than expected distribution of 10-fold or higher variants, the analyzed sample may be indicated as deviating from the expected distribution.

The sequencing data generated by sequencing the biological sample may analyzed to characterize any copy number variation after being normalized using the normalization information. It should be understood that the biological sample sequencing data and the baseline sequencing data may be in the form of raw data, base call data, or data that has gone through primary or secondary analysis.

Further, it should be understood that CNVs may be identified as being part of a gene, an intragenic region, etc. It should also be understood that CNV detection may be associated with duplicate or deleted sequences. Accordingly, CNV detection may represent duplicate copies of a nucleic acid region, such as a region including one or more genes. In one embodiment, CNVs are duplicate or deleted genomic regions of at least 1kb in size.

Sequencing coverage describes the average number of sequencing read counts that align to, or “cover,” known reference bases. The coverage level often determines whether variant discovery can be made with a certain degree of confidence at particular base positions. At higher levels of coverage, each base is covered by a greater number of aligned sequence reads, so base calls can be made with a higher degree of confidence. Reads are not distributed evenly over an entire genome, simply because the reads will sample the genome in a random and independent manner. Therefore many bases will be covered by fewer reads than the average coverage, while other bases will be covered by more reads than average. This is expressed by the coverage metric, which is the number of times a genome has been sequenced (the depth of sequencing). For targeted resequencing, coverage may refer to the amount of times a region is sequenced. For example, for targeted resequencing, coverage means the number of times the targeted subset of the genome is sequenced. The disclosed embodiments address noise in sequencing coverage due to bias.

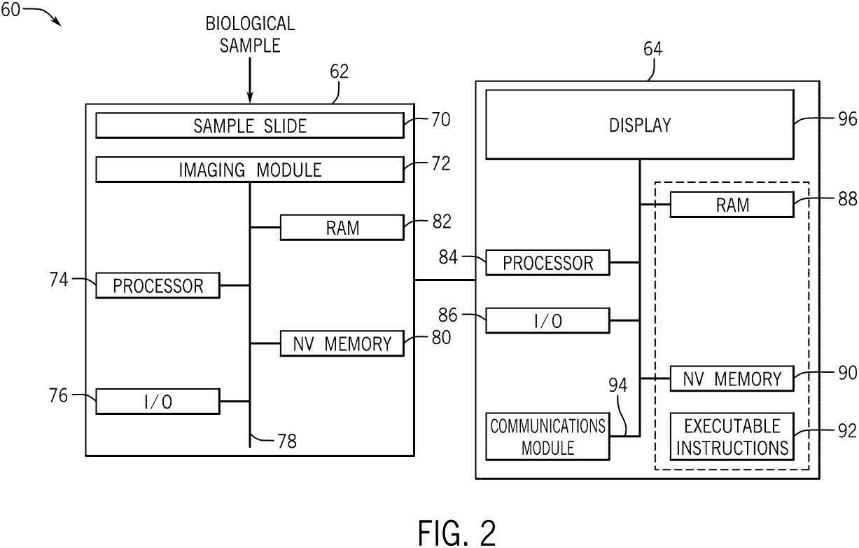

FIG. 2 is a schematic diagram of a sequencing device 60 that may be used in conjunction with the steps of the flow diagram of FIG. 1 for acquiring sequencing data (e.g., test sample sequencing data, baseline sequencing data) this is used for assessing copy number variation. The sequence device 60 may be implemented according to any sequencing technique, such as those incorporating sequencing-by-synthesis methods described in U.S. Pat. Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device 60. Such techniques use DNA ligase to incorporate oligonucleotides and identify the incorporation of such oligonucleotides and are described in U.S. Pat. No. 6,969,488; U.S. Pat. No. 6,172,218; and U.S. Pat. No. 6,306,597; the disclosures of which are incorporated herein by reference in their entireties. Some embodiments can utilize nanopore sequencing, whereby target nucleic acid strands, or nucleotides exonucleolytically removed from target nucleic acids, pass through a nanopore. As the target nucleic acids or nucleotides pass through the nanopore, each type of base can be identified by measuring fluctuations in the electrical conductance of the pore (U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties). Yet other embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides, or with zeromode waveguides as described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, the sequencing device 16 may be a HiSeq, MiSeq, or HiScanSQ from Illumina (La Jolla, CA).

In the depicted embodiment, the sequencing device 60 includes a separate sample processing device 62 and an associated computer 64. However, as noted, these may be implemented as a single device. Further, the associated computer 64 may be local to or networked with the sample processing device 62. In the depicted embodiment, the biological sample may be loaded into the sample processing device 62 as a sample slide 70 that is imaged to generate sequence data. For example, reagents that interact with the biological sample fluoresce at particular wavelengths in response to an excitation beam generated by an imaging module 72 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imaging module 72.

The imaging module detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imaging module 72 may be under processor control, e.g., via a processor 74, and the sample receiving device 18 may also include I/O controls 76, an internal bus 78, non-volatile memory 80, RAM 82 and any other memory structure such that the memory is capable of storing executable instructions, and other suitable hardware components that may be similar to those described with regard to FIG. 2. Further, the associated computer 20 may also include a processor 84, I/O controls 86, a communications module 84, and a memory architecture including RAM 88 and non-volatile memory 90, such that the memory architecture is capable of storing executable instructions 92. The hardware components may be linked by an internal bus 94, which may also link to the display 96. In embodiments in which the sequencing device is implemented as an all-in-one device, certain redundant hardware elements may be eliminated.

The present techniques facilitate detecting or calling CNVs in biological samples (e.g., tumor samples) without first normalizing the sequencing data to matched sequencing data. The technique uses a preprocessing step to generate a manifest file and a baseline file, which are used as input parameters for the normalization step. The manifest file and the baseline file are generated independent of and prior to analysis of a sample of interest to determine copy number variation. The manifest file and the baseline file are generated from non-matched samples (i.e., non-matched normal samples) and are determined via the baseline generation technique as provided herein. Baseline generation may be performed on the non-matched normal samples and the results of the baseline generation stored as baseline information (or normalization information) for access by executable instructions of the normalization technique. For example, a user with a sample of interest may perform analysis of one or more CNVs. In certain embodiments, after generation and storage, the baseline information is used in the analysis of a plurality of samples of interest at different and/or subsequent time points. The user may access the stored files based on the sequencing panel that corresponds to the baseline information.

In one embodiment, the copy number normalization information, once generated, is fixed for a particular sequencing panel. That is, the copy number normalization information is associated with the particular probes of the sequencing panel and is stored by the provider and sent to the user of the particular sequencing panel. Different sequencing panels have different copy number normalization information. In another example, a CNV-calling software package may store a plurality of different copy number normalization information, each associated with different sequencing panels. The user may select the appropriate normalization information based on the sequencing panel used to acquire the sequencing data. Alternatively, the sequencing device 60 may automatically acquire the appropriate copy number normalization information based on information input by the user related to the sequencing panel used. The CNV-calling software package may also be capable of receiving updates from a remote server if the copy number normalization information is refined by the provider.

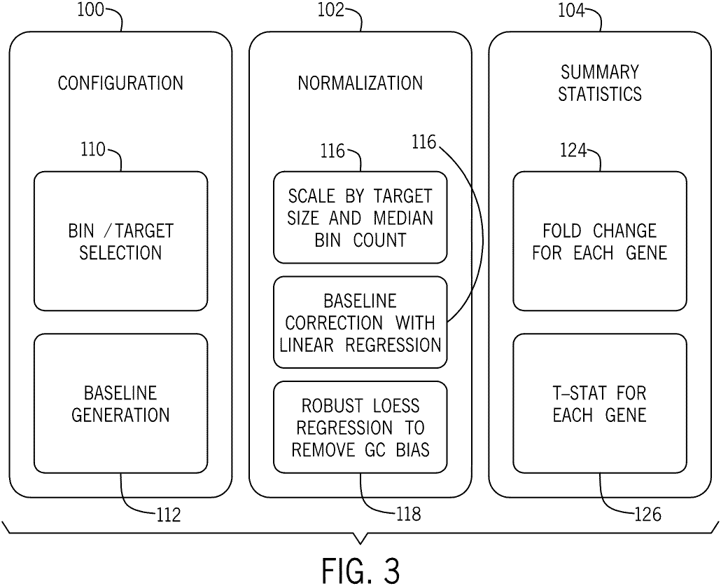

The problem of somatic copy number variation detection is solved by identifying representative baseline coverage behavior using a hierarchical clustering method and then leveraging linear regression and Loess regression for data normalization, as summarized in FIG. 3. The technique includes configuration 100 (e.g., algorithm training), normalization of samples of interest 102, and providing outputs or statistics 104, such as copy number fold changes and T-stats on an individual gene basis. For example, FC is the ratio between the median value of the gene of interest and genome median. T-stat may be the bin count distribution of the gene of interest compared to the rest of the genome (e.g., for a diploid organism).

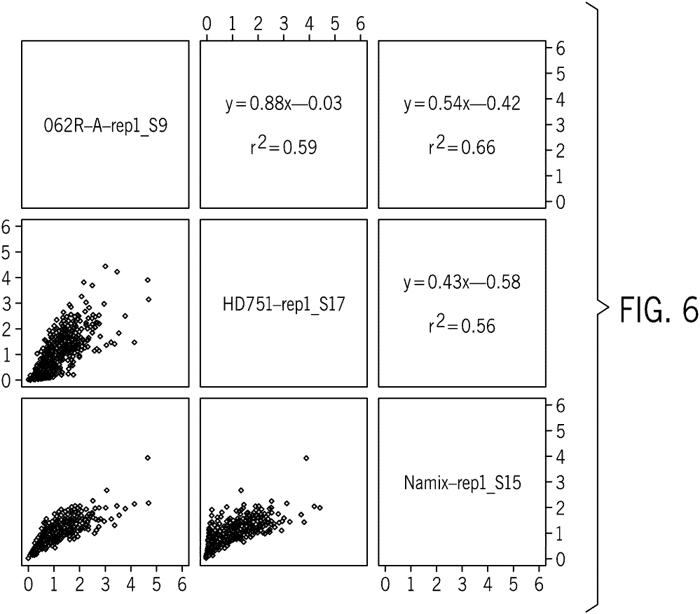

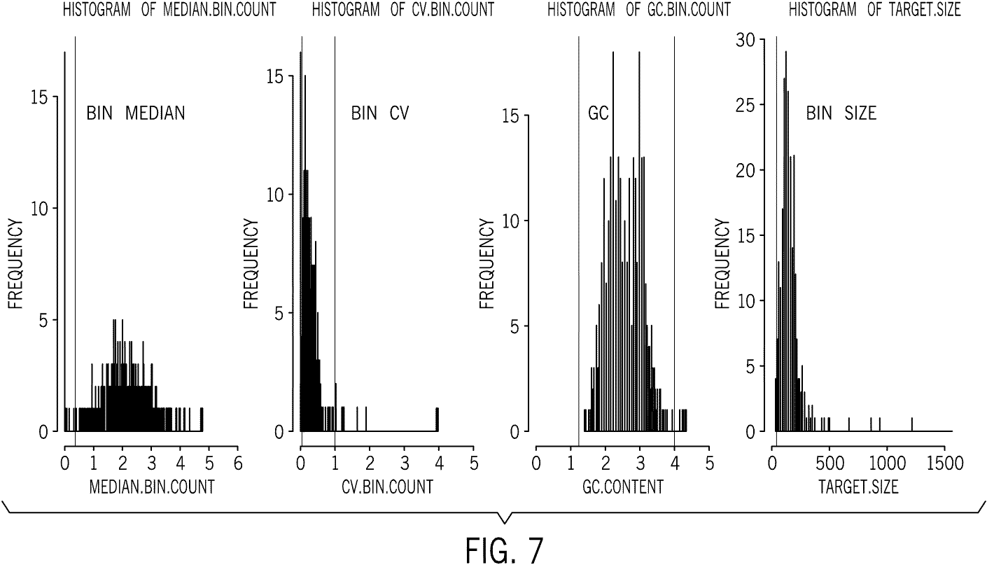

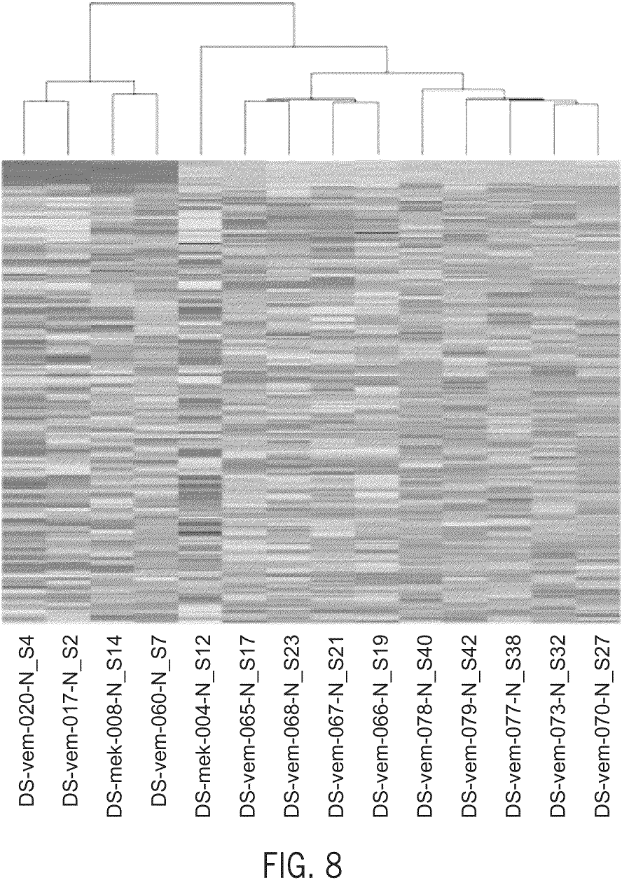

The preprocessing (algorithm training) may include the following steps:
1. Bin/exon selection 110: from a set of training normal samples (e.g., FFPE normal samples), calculate median, median absolute deviation, GC content and size for each bin (see FIG. 7). Then, bins with low median, large MAD, extreme GC content and small size are marked as bad bins in the manifest file. Only a small percentage of bins are affected by this step (~5%). For example, as shown in FIG. 6, filtering parameters used are
   Median > 0.25
   CV: (0.2)
   GC: (0.25, 0.8)
   Target size: >20bp
2. Baseline generation 112 from baseline or normal samples (e.g., FFPE normal samples): samples from different tissue types or with different DNA quality can have very different baseline behavior. Therefore, multiple baselines are used to correct the baseline effect. In one example, 4-5 normal FFPE samples from each tissue type are used to determine the median behavior for each bin to represent different tissue types. To generate baseline, hierarchical clustering is used to identify representative groups that reflect multiple underlying coverage behaviors in normal sample population. See FIG. 8. Clustering is correlated to sample quality. Once clusters are identified, the median value for each bin is used to create a baseline file that will be used for subsequent normalization. That is, the median bin count in each cluster is taken as baseline. By using a clustering method, the most “representative” behavior in normal samples is used for downstream normalization.

After the baseline or normalization (applied to assessed samples) using the reference baseline generated above, where the new sample is scaled to the normalization information by target size and median bin count 114.

1. Baseline correction 116: for a new sample, model its bin count as a linear combination of baselines: Y~c1+c2+c3. Due to potential CNVs in the new sample, outliers are first removed from Y, and the linear model is built on outlier removed values. In certain embodiments, outliers are masked. In other embodiments, only extreme outliers are removed or masked. Then, the ratio of Y and linear model prediction is used as baseline corrected value. Bin counts above or below 3 standard deviation are considered outliers.
Lm(Y[good.idx] ~ c1[good.idx] + c2[good.idx] + c3[good.idx])
Y_new ~ Y/predict (1 m, data=ALL)

2. Robust loess regression 118 to remove GC bias after step 1.

3. For each gene, calculate its fold change 124 by comparing its median bin value to the genome median. Additional statistics, e.g., t-stat for each gene 126, may also be determined.

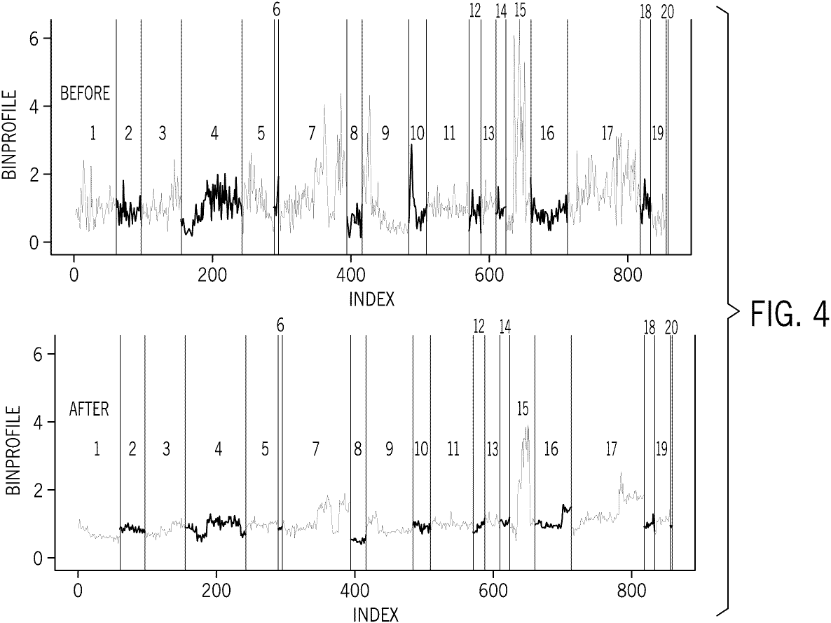

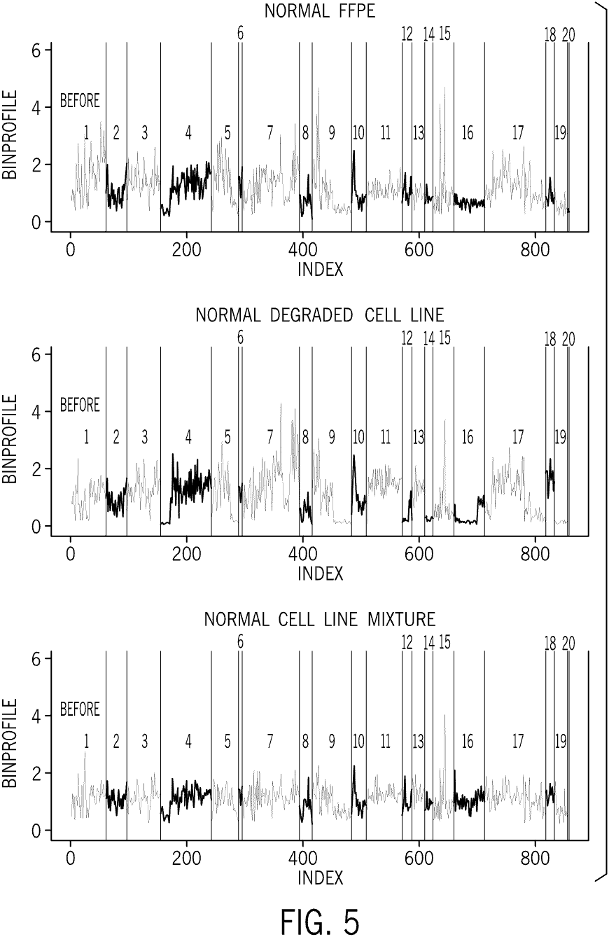

FIG. 4 shows bin profile data for sequencing results before and after the normalization, as provided herein, across a number of bins. The noise present in the “before” results is reduced as shown in the “after” results. The noise prevents accurate calling of copy number variants. FIG. 5 shows noise present in normal FFPE samples relative to a highly degraded cell line and a normal cell line mixture. The noise present in the data interferes with accurate CNV calling. Further, the noise is present in samples of varying quality. However, baseline correlation is poor among different sample types. Accordingly, the present techniques permit user input of sample type to select the appropriate normalization information.

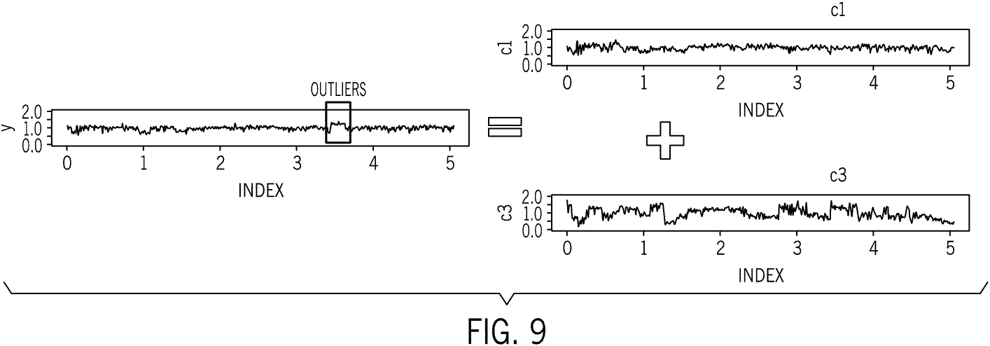

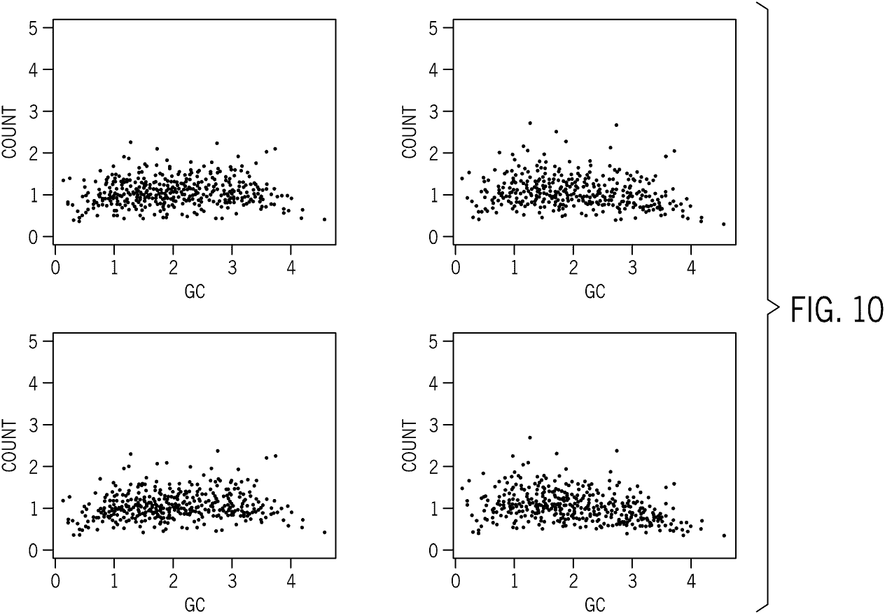

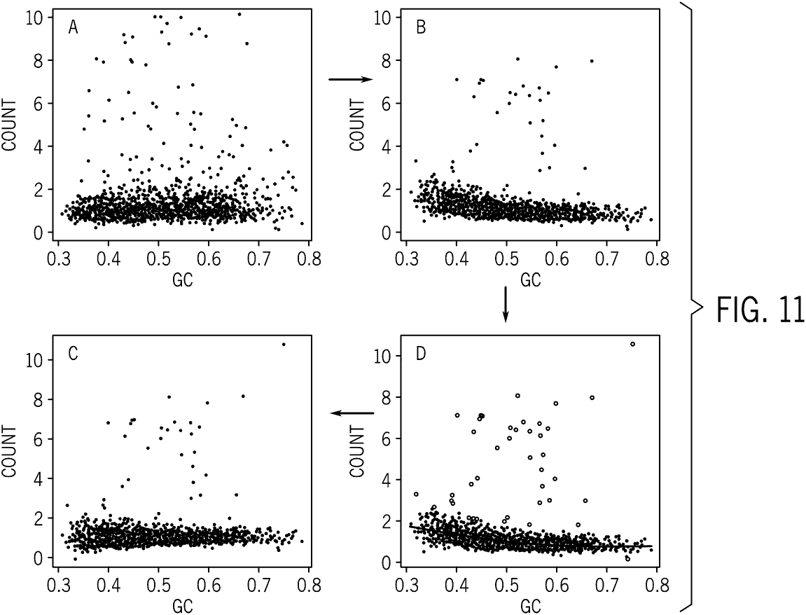

FIG. 9 shows the results of baseline correction with linear regression to remove noise, whereby c1 and c2 are two representative baselines learned from hierarchical clustering. As shown in FIG. 10, GC bias is sample specific. In general, extremely low GC or high GC regions are under-represented in reads. Some samples have more curvature than others. FIG. 11 is an illustration of normalization steps for step-wise approach. (A) due to the large baseline effect, there is no visible relationship between exon count and GC. (B) after baseline correction, there is a visiblie negative trend between count and GC. (C) Outliers are idenfied and loess regression is fitted on outlier removed data. (D) Final normalization results after remove GC bias.

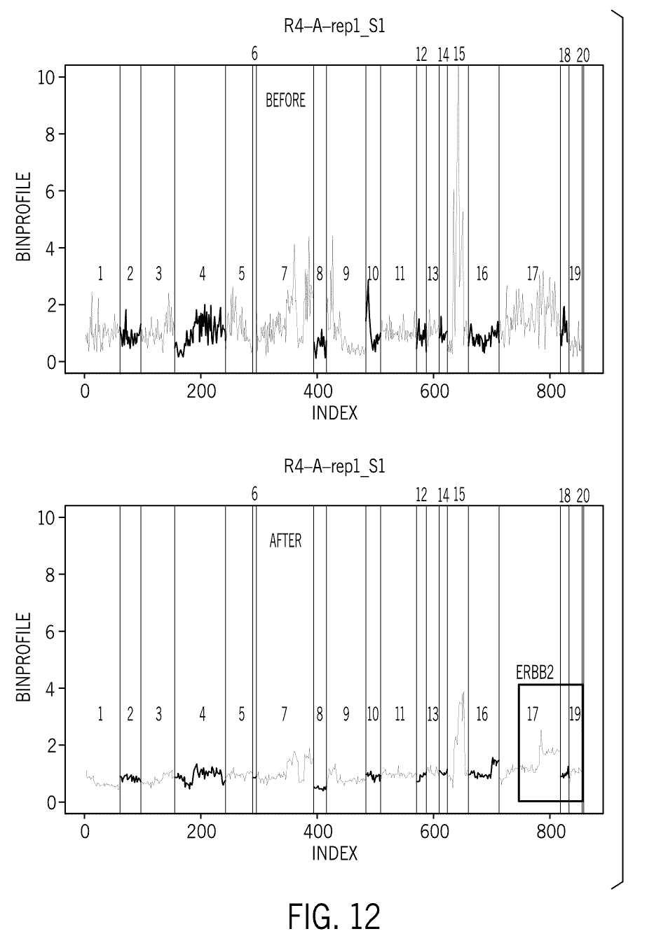

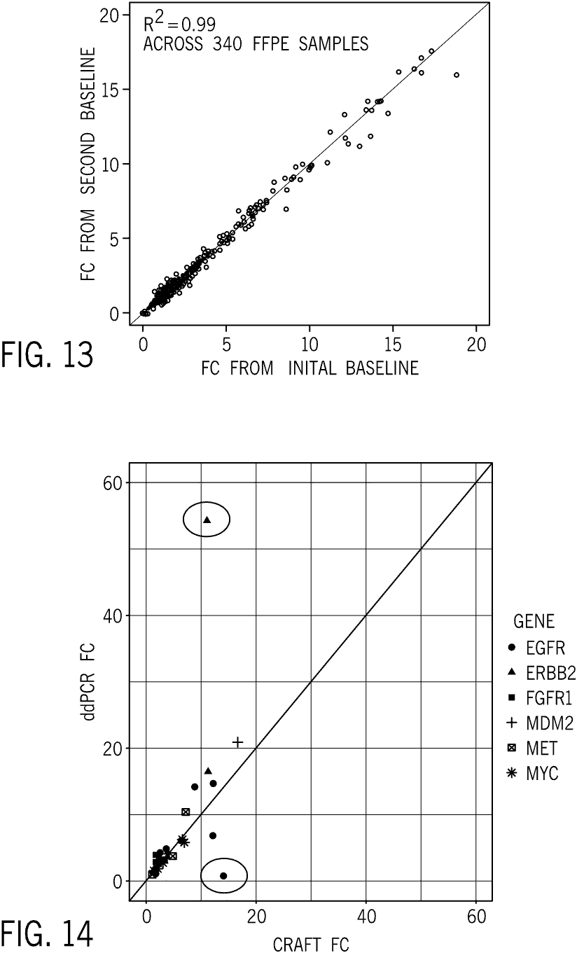

FIG. 12 shows before and after normalization results, including sequence bins for the ERBB2 gene. The “after” results demonstrate a significant reduction in noise via normalization as provided herein. FIG. 13 shows that the fold change detection is stable independent of baseline used, with R²=0.99 across 340 FFPE samples. FIG. 14 shows high concordance between the normalization techniques as provided herein and ddPCR across 22 FFPE samples tested using a panel for a number of regions of interest, including EGFR, ERBB2, FGFR1, MDM2, MET, and MYC.

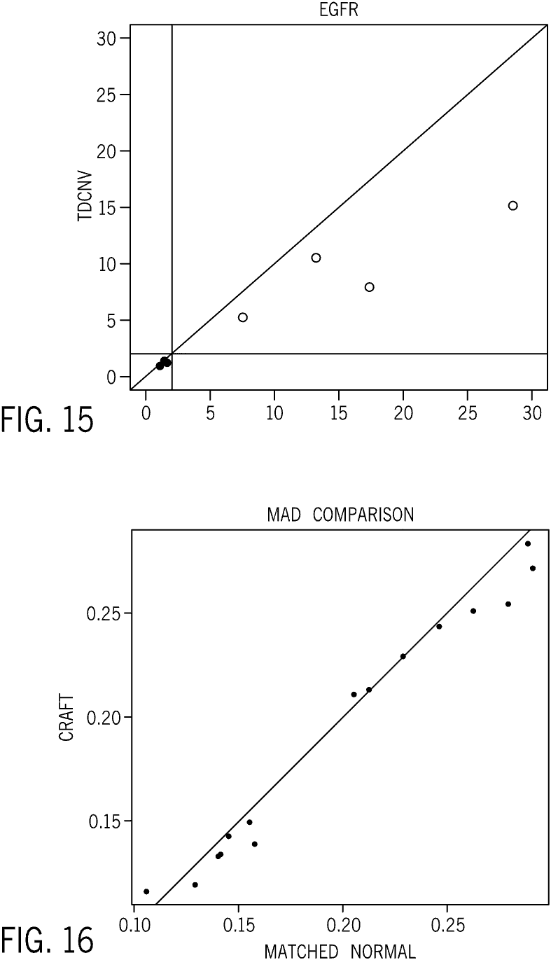

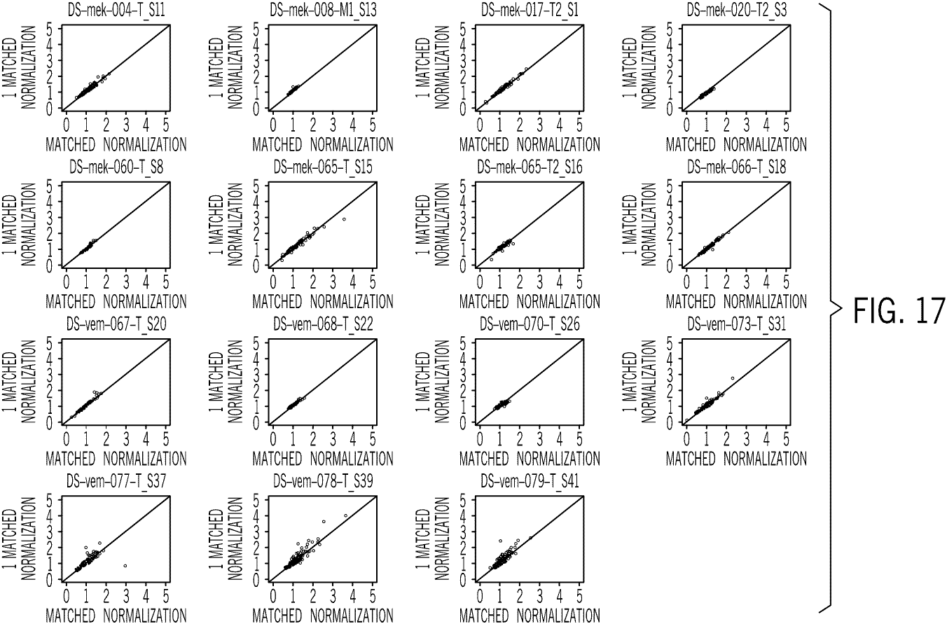

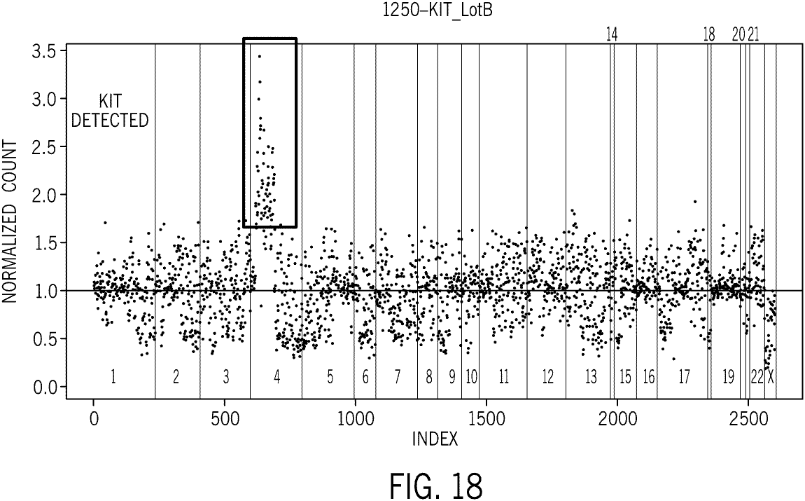

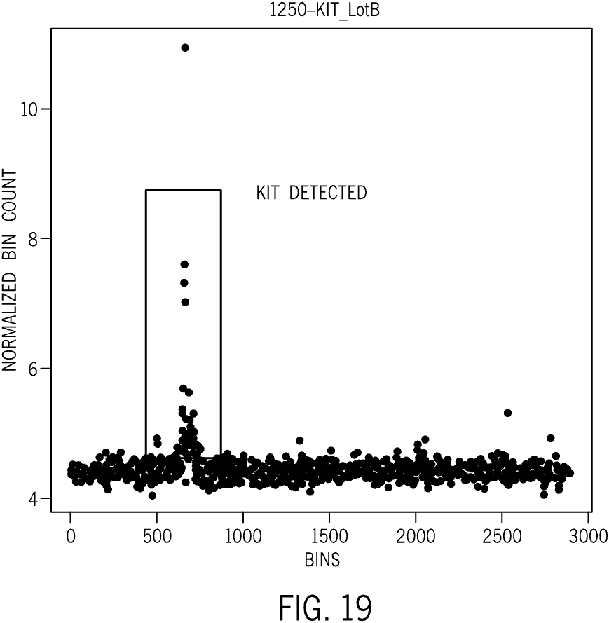

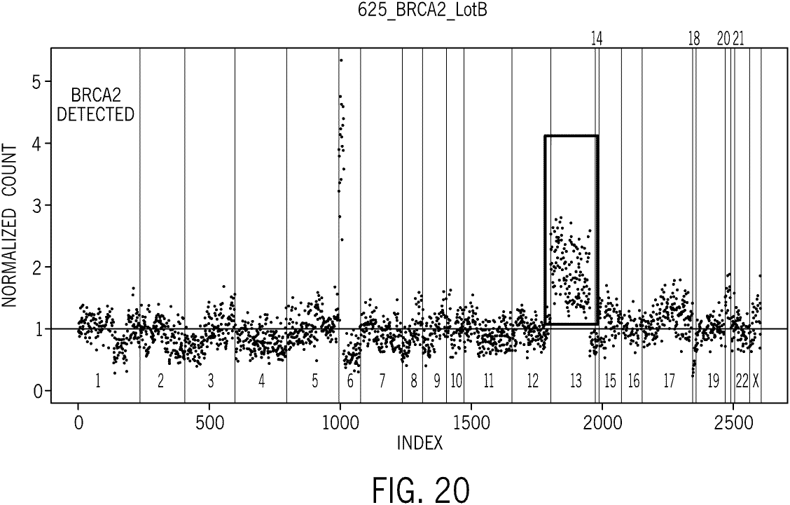

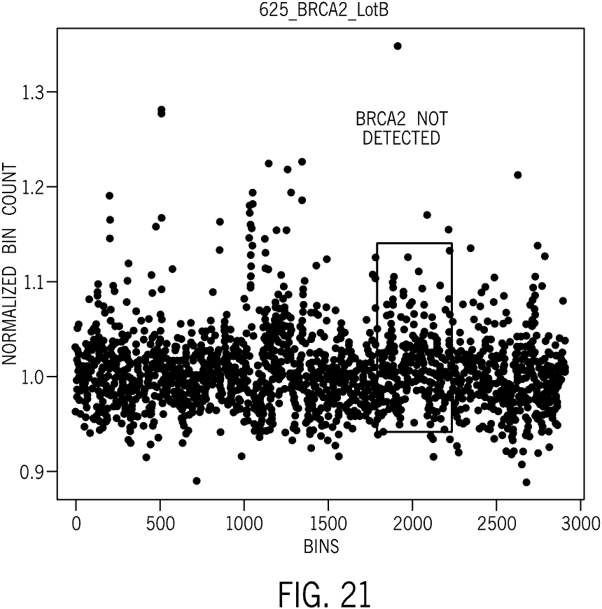

FIG. 15 is a comparison of the normalization technique used herein to baseline or control free method. The control free method doesn’t require any additional control or normal samples for normalization. It instead relies on the testing sample itself for data normalization. Compared to normalization technique used herein, control free method tends to underestaimte gene amplification level in terms of the measured fold change (FC) values. Addtionally, applying control free method on normal testing samples showed that the FC variability is much larger than the present normalization technique, which leads to a higher limit of bland (LoB). In general, control free method is both less sensitive and less specific than the normalization technique as provided herein. In FIG. 15, the Y-axis is a internal implementation of control free method, and X-axis is an embobiment of the normalization technique described herein. Compared to the normalization technique, control free method tends to underestimate fold change values.
FIG. 16 shows a median absolute deviation comparison of results using the normalization techniques as provided herein and matched normal samples with a paired t test p-value of 0.0202. FIG. 17 shows fold change comparison, with detected fold change (FC) comparison between the normalization techniques as provided herein (y-axis) and matched normal (x-axis);
FIGS. 18-21 show a comparison between the normalization techniques as provided herein and XHMM, a CNV method based on machine learning PCA approach, which doesn’t require matched normal samples. After data normalization, it employs a segmentation method to call CNVs within sample. The results shown for XHMM were obtained using the downloaded program run on the 15 CNV samples and compared to the normalization techniques. XHMM detected 10 out of 15 amplifications, whereas the normalization techniques detected 14 out of 14 CNVs with 1 no call. Based on the results, the normalization techniques have better sensitivity than XHMM.

The present techniques do not use or require matched normal samples to perform normalization. Instead, the normalization techniques herein use non-matched normal samples to generate reference baselines from which fold changes are detected. In certain embodiments, a plurality of normal samples are used to determine the reference baselines, and clustering of sequencing data of the plurality of samples is performed to determine the most representative normal bins. Accordingly, the reference baseline values are assessed on a per bin basis and not on a per sample basis. In addition, the present techniques incorporate more than one baseline behavior value in historical normal samples. The present techniques leverage linear regression for baseline correction, and Loess for GC correction. Results achieved include 100% sensitivity in R2 DVT study (including certain no-calls).

In comparison to other techniques, the normalization as provided yields better performance than control free in terms of LoB and LoD. Further, normalization is more economical relative to techniques using matched normal that require additional sample processing. CNV calling using normalization is more economical because the sequencing costs do not include costs for sequencing of matched normal samples. Accordingly, the sequencing run and operation of the sequencing device is more efficient. Other approaches, such as reference free approaches, do not yield high quality results due to probe pull down effects. Statistical techniques that use SVD decomposition or PCA also do not yield high quality results and/or have limited applicability for certain sample types.

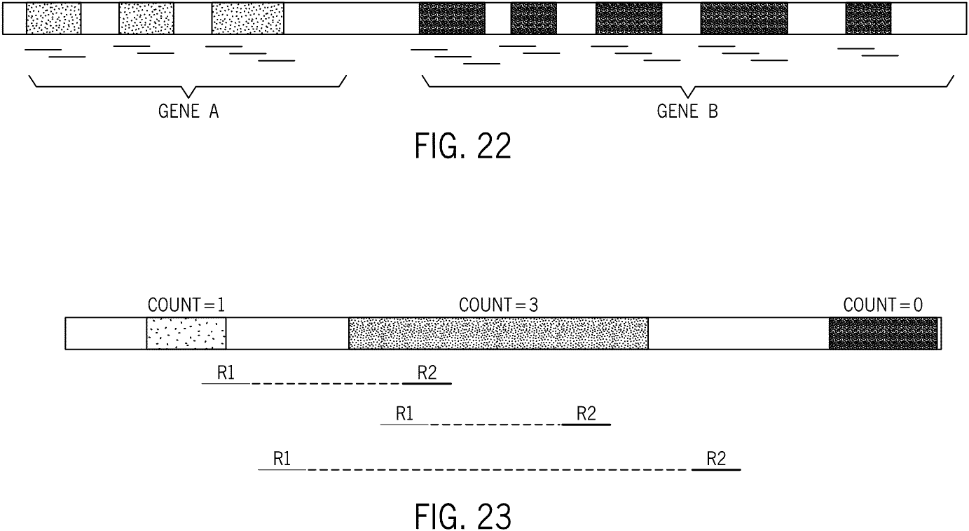

In particular embodiments, a bin as provided herein refers to a contiguous nucleic acid region of interest of a genome. A bin may be an exonic, intronic, or intragenic. Bins or bin regions may include variants, and, therefore, generally refer to the location or region of the genome rather than a fixed nucleic acid sequence. Bin counting is done at the fragment level, not the read level. For example, genes A and B, as shown in FIG. 22, may have various probes that target individual bins (shaded areas). FIG. 23 is a schematic representation of bin counts based on fragments, not reads. Fragments that overlap with a bin contribute to the bin count for that bin. A single fragment may contribute to the bin count for multiple bins. Accordingly, for each fragment, all targets it overlaps are found. Read filtering is performed to determine properly aligned pairs, non-PCR duplicates, positive strands (to avoid double counting), and MAPQ>20.

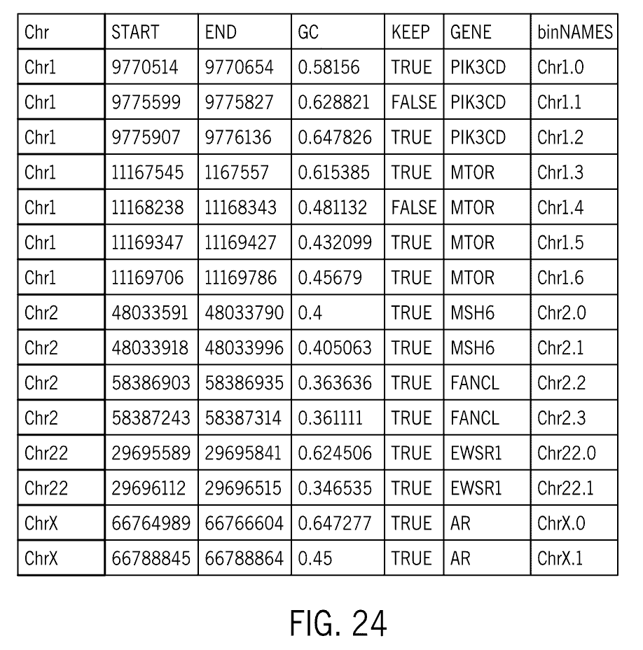

In certain embodiments, probe target selection may be improved to reduce the introduction of noise in the sequencing data. For example, in one technique, the probe selection may occur as outlined: for each gene, identify the number of targets with GC content between 0.3 and 0.8. If the number is smaller than 20, identify regions for not covered by current probe design. Create equally spaced windows of size 140bp and compute the GC and mappability (75mer) for each window. Select the top K windows by mappability and GC content. For the Y chromosome, which is used for gender classification, randomly select 40 regions with mappability of 1 and GC between 0.4 and 0.6. FIG. 24 is table of example bin designations and characteristics, indicating start and end sites for examined bins, GC content, and determined quality for certain genes.

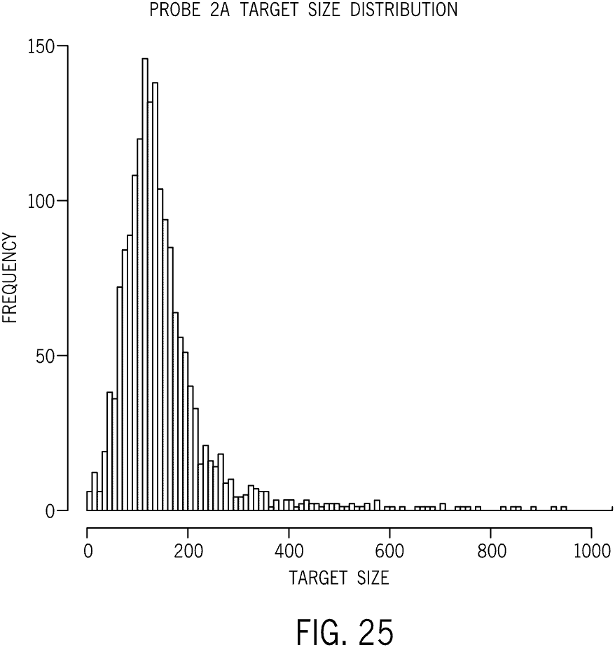

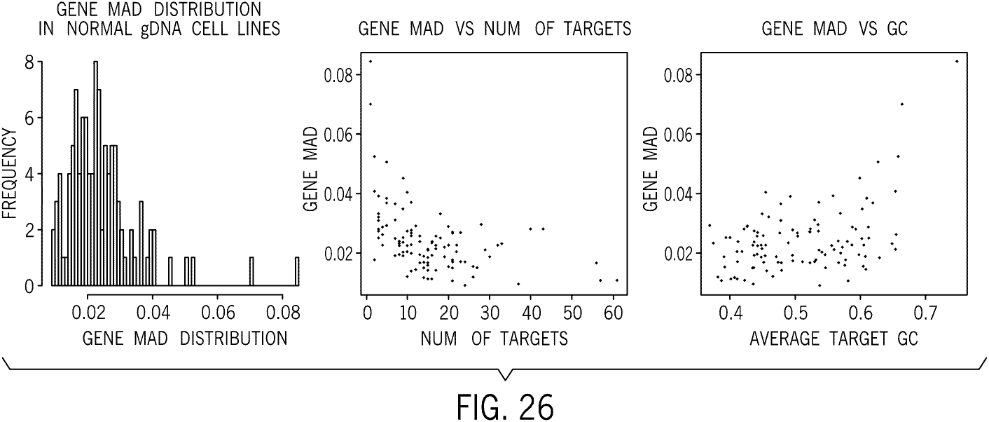

FIG. 25 is a plot of target size distribution for a probe. FIG. 26 shows gene median absolute distribution and comparison to number of targets and GC content of targets. In one embodiment, 20 good targets (30 - 80% GC) is sufficient to stabilize gene MAD in gDNA samples (middle plot).

In one example, 116 out of 170 genes in probe set 2C have fewer than 20 targets. 1042 additional targets are selected. 31 out of 49 amp genes have fewer than 20 targets. 350 additional targets are selected. For the Y chromosome, 40 targets are selected for gender classification. In sum, to cover all the 49 amp genes with at least 20 targets/gene, add 390 additional targets (140bp windows) to probe set 2C. FGF4, CKD4 and MYC still have less than 20 targets due to small gene size. Gene targets for certain genes are shown in Table 2.

TABLE 2

Gene targets

| Gene | CEBPA | FGF4 | FOXL2 | CDK4 | MYC | CD79B | HRAS | CD79A | VHL |
|---|---|---|---|---|---|---|---|---|---|
| Targets | 8 | 9 | 10 | 12 | 15 | 16 | 16 | 17 | 18 |

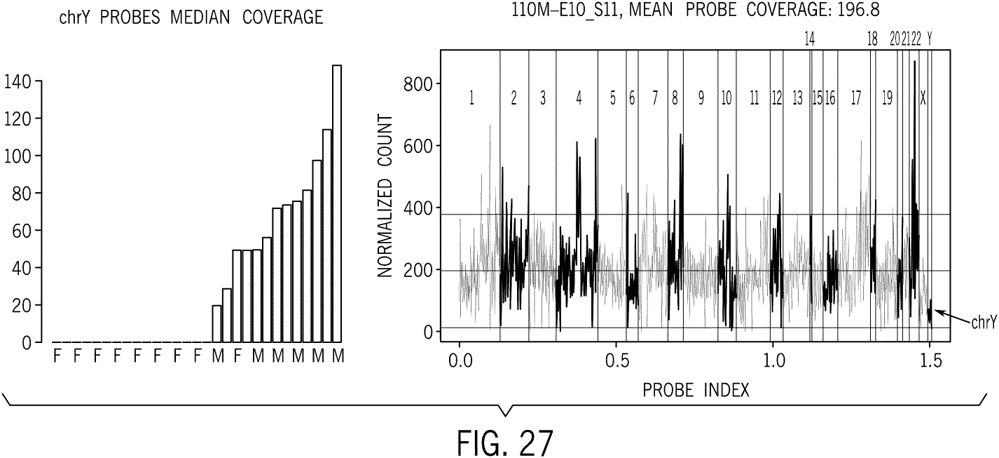

FIG. 27 shows gender classification of 29 FFPE samples and presence of chromosome Y coverage. Chromosome Y is indicated by the arrow in the right plot.

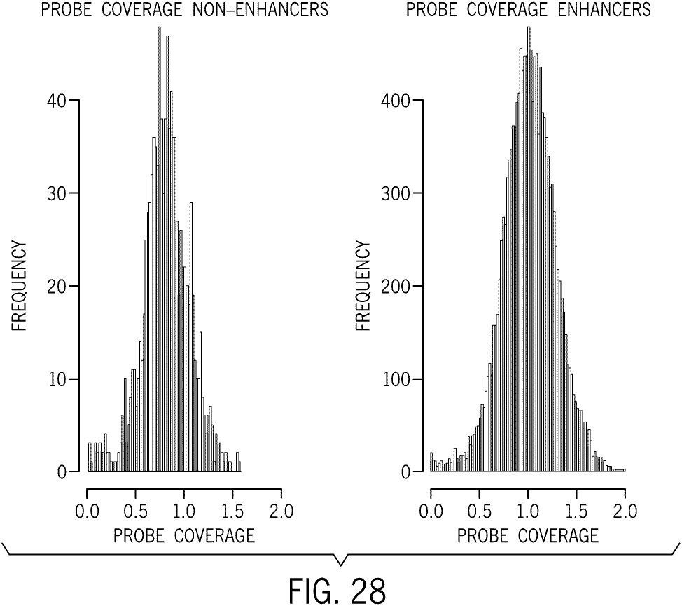

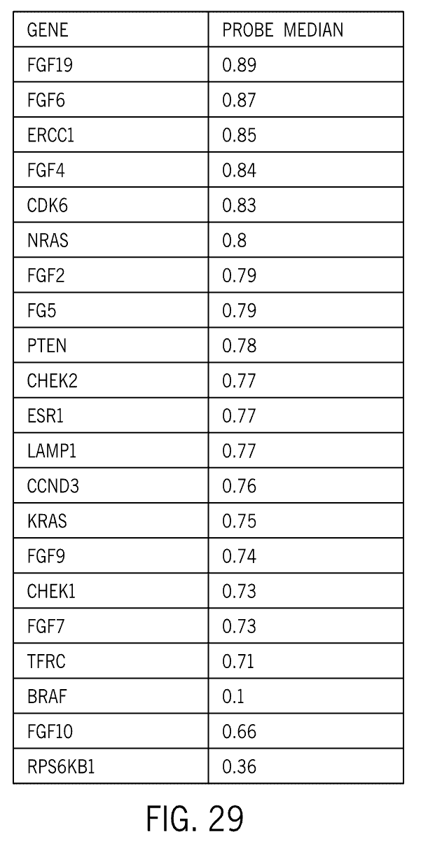

FIG. 28 shows a comparison of probe coverage with and without coverage enhancers; FIG. 29 shows a summary of probe coverage for a variety of genes;

Embodiments of the disclosed techniques include graphical user interfaces for displaying copy number variation information and that provide outputs or indications use and/or receive user input. FIG. 30 is an example of a graphical user interface 200. Execution of the normalization techniques, e.g., by a processor (see FIG. 2), cause CNV information to be displayed. The displayed CNV information, including the variant number along an axis, is post-normalization. That is, the copy number for the acquired sequencing data is analyzed for copy number variants after normalization has taken place. Accordingly, graphical user interface 200 displays normalized CNV information.

Technical effects of the disclosed embodiments include improved and more accurate determination of CNVs in a biological sample. Copy number variants may be associated with genetic disorders, cancer progression, or other adverse clinical conditions. Accordingly, improved CNV detection may permit sequencing data to provide richer and more meaningful information to clinicians. Further, the disclosed CNV assessment techniques may be used in conjunction with targeted sequencing techniques, which sequence only a portion of the genome. In this manner, CNVs may be identified from a more efficient sequencing strategy. The normalization techniques as provided herein address bias introduced into sequencing data that affects sequencing coverage counts.

While only certain features of the disclosure have been illustrated and described herein, many modifications and changes will occur to those skilled in the art. It is, therefore, to be understood that the appended claims are intended to cover all such modifications and changes as fall within the true spirit of the disclosure.

1. A method of normalizing copy number, comprising:
receiving a sequencing request from a user to sequence one or more regions of interest in a biological sample;
acquiring baseline sequencing data from the one or more regions of interest from a plurality of baseline biological samples that are not matched to the biological sample;
determining copy number normalization information using the baseline sequencing data, wherein the copy number normalization information comprises at least one copy number baseline for a region of interest of the one or more regions of interest; and
providing the copy number normalization information to the user.

2. The method of claim 1, wherein the baseline sequencing data comprises data representative of a sequencing read count for each bin of a plurality of bins, wherein each bin of the plurality of bins is associated with a respective region of interest.

3. The method of claim 2, wherein acquiring the baseline sequencing data comprises using a targeted sequencing panel and wherein the plurality of bins are defined using sequences corresponding to the regions of interest in the targeted sequencing panel.

4. The method of claim 2, wherein acquiring the baseline sequencing data comprises acquiring whole genome sequencing data.

5. The method of claim 2, wherein the sequencing read count is a measure of a number of individual sequencing reads in the baseline sequencing data corresponding to each bin.

6. The method of claim 3, comprising determining one or more of a median sequencing read count, median absolute deviation, GC content, and size for each bin of the plurality of bins.

7. The method of claim 6, comprising eliminating or masking bins from the plurality of bins with one or more of a low median, large median sequence coverage absolute deviation, GC content outside of a predetermined range, or a size below a size threshold from the baseline sequencing data before determining the copy number normalization information such that the copy number normalization information is determined using only remaining bins after the eliminating or the masking.

8. The method of claim 7, wherein eliminating or masking the bins comprises eliminating or masking bins with a median sequence coverage count of less than 0.25.

9. The method of claim 7, wherein eliminating or masking bins comprises eliminating or masking bins with a median sequence coverage with an absolute deviation above a threshold.

10. The method of claim 7, wherein e eliminating or masking bins comprises eliminating or masking bins with a GC content of less than 25% or greater than 80%.

11. The method of claim 7, wherein eliminating or masking bins comprises eliminating or masking bins with a target size of less than 20 bases.

12. The method of claim 2, comprising clustering the baseline sequencing data for each bin to determine the copy number baseline, wherein the copy number baseline is generated from a median sequencing read count per bin of the plurality of bins associated with the region of interest.

13. The method of claim 12, comprising determining copy number baselines for additional bins of the plurality of bins.

14. The method of claim 1, wherein the biological sample is a sample derived from an individual and wherein the plurality of baseline samples are from samples derived from different individuals.

15. The method of claim 1, wherein the biological sample is derived from a tumor tissue of an individual and wherein the plurality of baseline samples are derived from normal tissue that is not from the individual.

16. The method of claim 1, comprising receiving the sequencing data of the biological sample from the user, and determining that the sequencing data comprises a variation from the copy number baseline in the region of interest.

17. The method of claim 16, comprising generating an indication of the variation and providing the indication to the user.

18. The method of claim 17, wherein the indication is fold change in copy number of the biological sample relative to the copy number baseline for the region of interest.

19. The method of claim 16, comprising masking outlier bins in the sequencing data before determining that the sequencing data comprises the variation from the copy number baseline in the region of interest.

20. The method of claim 19, comprising applying loess regression to the sequencing data to eliminate GC bias after masking the outlier bins.

21. The method of claim 19, comprising fitting the sequencing data to a curve after masking the outlier bins.

22. The method of claim 1, wherein the sequencing data is acquired using an exome sequencing panel.

23. The method of claim 1, wherein providing the copy number baseline information to the user comprises providing information representative of hypothetical reference sample that mimics a matched sample for the user and that is not generated using matched samples.

24. A method of detecting copy number variation, comprising:
acquiring sequencing data from a biological sample, wherein the sequencing data comprises a plurality of raw sequencing read counts for a respective plurality of regions of interest;
normalizing the sequencing data to remove region-dependent coverage bias, wherein the normalizing comprises:
for each region of interest, comparing a raw sequencing read count of one or bins in a region of interest of the biological sample to a baseline median sequencing read count to generate a baseline-corrected sequencing read count for the one or more bins in the region of interest, wherein the baseline median sequencing read count for one or more bins in the region of interest is derived from a plurality of baseline samples that are not matched to the biological sample and is determined from only the most representative portions of the baseline sequencing data for each region of interest; and
removing GC bias from the baseline-corrected sequencing read count to generate a normalized sequencing read count for each region of interest; and
determining copy number variation in each region of interest based on the normalized sequencing read count of the one or more bins in each region of interest.

25. The method of claim 24, wherein each region of interest comprises a single bin.

26. The method of claim 24, wherein each region of interest comprises a plurality of bins, and wherein the baseline median sequencing read count is a median across the plurality of bins.

27. The method of claim 24, wherein the method does not comprise acquiring sequencing data from a matched biological sample.

28. The method of claim 24, wherein the method is control free.

29. The method of claim 24, comprising determining a clinical status of the biological sample based on the copy number variation in each region of interest.

30. The method of claim 29, wherein the biological sample is a somatic sample and wherein the clinical status comprises a designation of tumor or normal.

31. The method of claim 24, wherein the baseline median sequencing read count for each region of interest is determined by clustering the baseline sequencing data.

32. The method of claim 32, wherein a first baseline median sequence coverage count for a first region of interest is derived from a first subset of the plurality of baseline samples and wherein a second baseline median sequence coverage count for a second region of interest is derived from a second subset of the plurality of baseline samples that is different from the first subset.

33. The method of claim 24, comprising removing or masking outlier bins in the sequencing data before normalizing the sequencing data.

34. The method of claim 24, wherein normalizing the sequencing data comprising applying loess regression to the sequencing data fit the sequencing data to a curve after removing or masking the outlier bins.

35. The method of claim 24, wherein the region-dependent bias comprises one or more of GC bias, PCR bias, or DNA quality bias.

36. A method of assessing a targeted sequencing panel, comprising:
identifying a first plurality of targets in a genome for a targeted sequencing panel, wherein the first plurality of targets corresponds to portions of a respective plurality of genes;
determining a GC content of each of the first plurality of targets;
eliminating targets of the first plurality of targets with GC content outside of a predetermined range to yield a second plurality of targets smaller than the first plurality of targets;
when, after the eliminating, the an individual gene has fewer than a predetermined number of targets corresponding portions to the individual gene, identifying additional targets in the individual gene;
adding the additional targets to the second plurality to yield a third plurality of targets; and
providing a sequencing panel comprising probes specific for the third plurality of targets.
